(19) United States
(12) Patent Application Publication
(10) Pub. No.: US 2022/0362472 A1
(43) Pub. Date: Nov. 17, 2022

(54) DRUG DELIVERY DEVICE WITH TEMPERATURE MEASUREMENT AND RELATED METHOD

(71) Applicant: AMGEN INC., Thousand Oaks, CA (US)

(72) Inventors: Azita Rahbari, Cestlake Village, CA (US); Sunitha Dasoju, Ventura, CA (US); Antonio Antonini, Milano (IT); Nicola Antonio Alagia, Milano (IT); Angelo Tosarini, Milano (IT)

(21) Appl. No.: 17/630,207

(22) PCT Filed: Jul. 29, 2020
(86) PCT No.: PCT/US20/43946

§ 371 Date: Jan. 26, 2022

Related U.S. Application Data
(60) Provisional application No. 62/881,657, filed on Aug. 1, 2019.

(51) Int. Cl. A61M 5/24

(52) U.S. Cl. CPC A61M 5/24; A61M 2005/2414; A61M 2205/3368

(57) ABSTRACT

A drug delivery device and related method of operation of a drug delivery device is described that includes a housing, a reservoir removably disposed within the housing and having a sidewall defining an interior for containing a drug, a temperature sensor that is configured to measure a current temperature relating to at least one of the reservoir, the sidewall of the reservoir, the housing, or the drug, and an output device that is configured to output data relating to the current temperature.

### CROSS-REFERENCE TO RELATED APPLICATION

This application claims the benefit of U.S. Provisional Application No. 62/881,657, filed on Aug. 1, 2019, which is hereby incorporated by reference herein in its entirety.

### FIELD OF DISCLOSURE

The present disclosure generally relates to a drug delivery system and, in particular, to a drug delivery system including temperature measurement.

### BACKGROUND

Drugs can be administered through the use of drug delivery devices such as autoinjectors or on-body injectors. Autoinjectors and on-body injectors may be used to help automate the injection and delivery or administration process, thereby simplifying the process for certain patient groups or sub-groups for which use of the syringe/vial combination or pre-filled syringe systems would be disadvantageous, whether because of physiological or psychological barriers, form factors, or ergonomic considerations.

Patients commonly receive drugs to treat a wide variety of medical conditions, which can be administered via injection or infusion. These injections or infusions may include intradermal, subcutaneous, intramuscular, intravenous, and intraperitoneal methods. Typically, injections or infusions involve the use of a hollow cannula or needle through which the drug passes from a container to the patient.

With regard to the subcutaneous and intramuscular injection routes, considerable attention has been devoted to providing a reproducible motion relative to the insertion of the cannula or needle through the skin to position the needle at a proper distance into the body, and then to provide a reproducible rate of delivery through the cannula or needle into the patient. Very often, providing a reproducible rate of delivery involves providing a reproducible motion for the movement of a plunger along the inside of a syringe or cartridge. Various mechanisms have been designed for controlled release of stored energy to advance the needle into the patient, and then to advance the plunger relative to the bore of the syringe or cartridge. Springs, motors, chemical reactions, and phase-changing materials have all been considered to provide the motive force for advancement of the needle and/or the plunger. Reproducible motion is considered fundamental to predictable drug delivery.

The temperature, and resulting viscosity, of the drug can also affect operation of the drug delivery components and the resulting drug delivery time. Refrigeration is required for some drugs and patients are instructed to wait a predetermined amount of time after the drug is removed from the refrigerator before performing an injection. This wait time is intended to reduce the viscosity of the drug and reduce discomfort during the injection. Other than this instruction, however, the patient is provided with no feedback to indicate that drug has indeed reached a desired temperature and is ready for injection.

### SUMMARY

In accordance with a first aspect, a drug delivery device is disclosed that includes a housing, a reservoir removably disposed within the housing and having a sidewall defining an interior for containing a drug, a temperature sensor that is configured to measure a current temperature relating to at least one of the reservoir, the sidewall of the reservoir, the housing, or the drug, and an output device that is configured to output data relating to the current temperature.

According to some forms, the drug delivery device can include a controller that is coupled to the temperature sensor and the output device, where the controller can be programmed to compare the current temperature to a predetermined target temperature. In further forms, the controller can be further programmed to receive and/or output data relating to the current temperature and the predetermined target temperature and/or to provide a signal to a user in response to determining that the current temperature corresponds to the predetermined target temperature.

According to some forms, the drug delivery device can include a drug data portion associated with the reservoir. In further forms, the drug delivery device can include a reader that is configured to read the drug data portion to determine injection data associated with the drug and, optionally, a controller coupled to the reader and the temperature sensor, where the controller is programmed to receive input from the reader and to compare the current temperature to a predetermined target temperature from the reader. In some versions, the drug data portion can include a machine readable code; and the reader can be a scanner configured to read the machine readable code.

According to some forms, the housing can include a bay that is configured to removably receive the reservoir therein. In further forms, the temperature sensor can be disposed adjacent to the bay and/or the housing can include a door movable between an open position and a closed position, where the door is configured to receive the reservoir in the open position and align the reservoir with the temperature sensor within the bay in the closed position.

According to some forms, the drug delivery device can further include one or more of the following aspects: a cassette configured to receive the reservoir therein, where the cassette defines a window providing access for the temperature sensor to measure the current temperature; the output device can include a display having a scale portion providing a visual indication of the current temperature; or the drug disposed in the reservoir.

In accordance with a second aspect, a method of delivering a drug product is disclosed that includes receiving a reservoir adapted to contain a drug within a drug delivery device, measuring a current temperature relating to at least one of the reservoir, a sidewall of the reservoir, a housing of the drug delivery device, or a drug contained within the reservoir with a temperature sensor, and outputting data with an output device of the drug delivery device relating to the current temperature.

According to some forms, the method can include reading a drug data portion associated with the reservoir with a reader of the drug delivery device to determine injection data associated with the drug contained within the reservoir. In a further form, the method can include determining an injection temperature for the drug associated with the injection data with a controller of the drug delivery device and determining whether the current temperature corresponds to the injection temperature with the controller. In some versions, the output device can include a display and outputting data can include displaying a visual indication of the current temperature in relation to the injection temperature on a scale portion of the display. In some versions, reading the drug data portion with the reader of the drug delivery device can include reading a machine readable code with a scanner.

According to some forms, receiving the reservoir within the drug delivery device can include receiving a cassette including the reservoir in a bay of an autoinjector device.

In accordance with a third aspect, a drug delivery system is disclosed that includes a container having a reservoir adapted to contain a drug and a drug data portion. The system further includes a drug delivery device having a bay configured to removably receive the container therein. The drug delivery device includes a temperature sensor disposed adjacent to the bay and configured to measure a temperature of the drug within the reservoir, a reader configured to read the drug data portion of the container to determine injection data associated with the drug contained within the reservoir, an output device, and a controller. The controller is coupled to the temperature sensor, the reader, and the output device, and is programmed to: determine an injection temperature for the drug associated with the injection data, determine whether the temperature of the drug corresponds to the injection temperature, and provide an indication to a user with the output device in response to determining that the temperature of the drug corresponds to the injection temperature.

According to some forms, the drug delivery system can include one or more of the following aspects: the temperature sensor can be a contactless temperature sensor; the temperature sensor can be an infrared temperature sensor; the output device can be a display including a scale portion providing a visual indication of the temperature of the drug in relation to the injection temperature; the drug data portion can be a machine readable code and the reader can be a scanner configured to read the machine readable code; the injection temperature can be an injection temperature range; the reservoir can include an opening, the container can further include a plunger-stopper moveable within the reservoir relative to the opening to force drug out of the reservoir through the opening, and the drug delivery device can include a plunger rod having a first end in contact with the plunger-stopper and an actuator coupled to the plunger rod; or the system can further include a drug disposed in the reservoir.

According to some forms, the container can be a pre-filled syringe, which can further include a needle in fluid communication with the reservoir. In further forms, the drug delivery device can be an autoinjector device, the container can be a cassette including a housing configured to receive the pre-filled syringe therein and defining a window providing access for the temperature sensor to determine the temperature of the drug; and/or the autoinjector device can include a door movable between an open position and a closed position, where the door is configured to receive the container in the open position and align the container with the temperature sensor within the bay of the autoinjector device in the closed position.

In accordance with a fourth aspect, a method of delivering a drug product is disclosed that includes receiving a container including a reservoir adapted to contain a drug within a bay of a drug delivery device, measuring a temperature of a drug contained within the reservoir with a temperature sensor of the drug delivery device, reading a drug data portion of the container with a reader of the drug delivery device to determine injection data associated with the drug contained within the reservoir, determine an injection temperature for the drug associated with the injection data with a controller of the drug delivery device, determine whether the temperature of the drug corresponds to the injection temperature with the controller of the drug delivery device and providing an indication to a user with an output device of the drug delivery device in response to determining that the temperature of the drug corresponds to the injection temperature

According to some forms, the method can include one or more of the following aspects: measuring the temperature of the drug within the reservoir of the container with the temperature sensor can include measuring a temperature of the drug with a contactless infrared temperature sensor; the drug can be contained within a pre-filled syringe received within a housing of a cassette and measuring the temperature of the drug can include measuring a temperature of the drug through a window defined by the housing with the temperature sensor; the output device can include a display and providing the indication to a user can include displaying a visual indication of the temperature of the drug in relation to the injection temperature on a scale portion of the display; reading the drug data portion of the container with the reader of the drug delivery device can include reading a machine readable code with a scanner; or receiving the container within the bay of the drug delivery device can include receiving the container in a door of an autoinjector device in an open position and aligning the container with the temperature sensor within the bay of the autoinjector device in a closed position.

### BRIEF DESCRIPTION OF THE DRAWINGS

The above needs are at least partially met through provision of the embodiments described in the following detailed description, particularly when studied in conjunction with the drawings, wherein:

FIG. 1 is a diagrammatic view of an autoinjector drug delivery device in accordance with various embodiments;

FIG. 2 is a side view of an embodiment of an autoinjector apparatus comprising a cassette and an autoinjector, showing the cassette prior to installation in the autoinjector.

FIG. 3A is a front view of the autoinjector apparatus of FIG. 2 showing the cassette installed in the autoinjector.

FIG. 3B is a side view of a first side of the autoinjector apparatus of FIG. 2 showing the cassette installed in the autoinjector.

FIG. 3C is a rear view of the autoinjector apparatus of FIG. 2 showing the cassette installed in the autoinjector.

FIG. 3D is side view of a second side of the autoinjector apparatus of FIG. 2 showing the cassette installed in the autoinjector.

FIG. 3E is an end view of a first end of the autoinjector of the autoinjector apparatus of FIG. 2.

FIG. 3F is an end view of a second end of the autoinjector of the autoinjector apparatus of FIG. 2.

FIG. 3G is a perspective view of the autoinjector apparatus of FIG. 2 showing a user interface indicating a temperature of a drug contained within the cassette installed therein.

FIG. 3H is a sectional side view of an embodiment of the autoinjector apparatus showing the cassette installed in the autoinjector.

FIG. 3I is a cross-sectional view of a portion of the autoinjector apparatus of FIG. 2 showing the cassette installed in the autoinjector and a temperature sensor.

FIG. 4 is an exploded perspective view of an embodiment of the cassette.

FIG. 5 is a sectional side view of an embodiment of a drug container that can be provided in the cassette.

FIG. 6 is a diagrammatic view of an on-body injector drug delivery device in accordance with various embodiments.

### DETAILED DESCRIPTION

A drug delivery system and method is provided that monitors the temperature of a drug and provides an indication when the drug has reached an optimal temperature/viscosity, which is based on the drug formulation's viscosity profile. The patient benefits of this system and method are twofold: an optimum viscosity is ensured to reduce injection discomfort and a wait time to perform an injection is minimized.

A drug may be injected or infused using a variety of different approaches, technologies, and systems. In one example, the drug may be filled into a reservoir in the form of a syringe or other appropriate primary container, e.g., a cartridge, and then the pre-filled syringe or other container may be combined with an autoinjector that may be used to automate the movement of a plunger within the bore of the syringe or container, and optionally the insertion of a cannula or needle into the patient. For example, the autoinjector may include a drive (e.g., a motor, spring(s), propellant reservoir, etc.) that causes the container to move within a housing and/or the plunger to move within the container upon manipulation of an actuator (e.g., depressing a button).

In whatever form the drug delivery system may take, it remains important to follow the appropriate storage recommendations for the drug to be injected or infused, because failure to follow these recommendations can result in subpotent or incomplete delivery of pharmaceutical products, and potentially therapeutic failure. For example, the storage recommendations for certain products require storage at low temperatures (2-8° C.), i.e., refrigeration.

Given the variety of different approaches, technologies, and systems for drug delivery, there are a number of different options for storage of the drug and the associated drug delivery device. For example, the drug may be refrigerated in its (primary) container or reservoir (e.g., pre-filled syringe, cartridge, etc.), while the associated drug delivery device (e.g., autoinjector) may be stored at room temperature, the reservoir being combined with the remainder of the drug delivery device at the time of use. Alternatively, the drug (positioned in its container or reservoir) and the associated drug delivery device may be refrigerated together. For example, the reservoir may be combined with the associated drug delivery device prior to or during refrigeration, such that the device is already assembled for use upon removal from storage. It is also possible that the drug-filled container and the drug delivery device may be disposed in the same packaging for storage (e.g., as a kit), but the drug-filled reservoir has not been disposed within the drug delivery device.

Storage of certain drug products (with or without the associated drug delivery device) at low temperatures may be important to prevent a subpotent product or incomplete or suboptimal delivery. For example, storage at low temperature may affect the physical characteristics of drug product or the action of the drug delivery device. Certain drug products exhibit increased viscosity at lower temperatures, which may inhibit delivery or make the rate of delivery less predictable soon after removal from the low temperature. Other drug products may become more viscous with increased temperature and therefore more difficult or less predictable to deliver the longer the drug is kept at room temperature. Further, storage at low temperatures may affect patient comfort when the drug is delivered. Certain patients will find administration of low temperature fluids to be painful. In addition, reductions in the rate of injection/infusion caused by low temperature effects on the drug and/or the device may be considered to be painful.

Once the drug product (and optionally the drug delivery device) is removed from storage, exposure to high temperatures may result in suboptimal drug delivery or delivery of a subpotent drug product. As noted above, depending on the storage recommendations, exposure to too high temperatures may require disposal of the drug product. Exposure to too high temperatures may also affect components of the drug delivery device, such as the battery.

This disclosure focuses on a drug delivery device that monitors the temperature of the drug and provides an indication that the drug is at a suitable temperature/viscosity for delivery. In some versions, the drug delivery device can identify or receive an identification of the particular drug within the device to thereby identify an optimal temperature/viscosity amount or range. The measured drug temperature is used to provide feedback to a user to enhance usability and reduce injection discomfort. The drug delivery device can also control operation based on determining that the drug is at the optimal temperature and associated viscosity. In some versions, the drug delivery device, which can be an autoinjector device, can utilize a built-in sensor to measure the drug temperature and notify a user when to perform an injection based on a viscosity profile and temperature for the particular drug.

Currently, patients are instructed to wait 30 minutes once a drug is removed from the refrigerator before performing an injection with the goal of reducing the viscosity of the drug and reducing potential injection discomfort associated with higher viscosities. With this arrangement, however, no feedback is provided to the patient to indicate that the drug has reached room temperature and is ready for injection. Additionally, based on some drug formulation's viscosity profiles, the drug may reach an optimal injection viscosity at a lower temperature than room temperature, which would reduce a wait time for the patient. With the drug delivery system provided herein that monitors the temperature of the drug, determines an injection temperature for the drug, and provides an indication to the patient when the drug has reached the injection temperature, the patient benefits are twofold: ensuring optimum viscosity is reached to reduce potential injection discomfort and minimizing wait time to perform an injection.

The autoinjector drug delivery device described herein is capable of sensing the drug temperature inside the pre-filled syringe. In one embodiment, the autoinjector drug delivery device includes an infrared sensor powered by a battery of the device. This contactless sensor is able to measure the temperature of an object in its field of view and removable a cassette or other container for the drug includes a window that allows the infrared light to measure the syringe temperature. The temperature sensor can be disposed adjacent to a needle of the syringe to accommodate temperature monitoring for all syringe types and fill volumes. A controller of the autoinjector drug delivery device can be programmed to monitor the drug/syringe temperature upon insertion of the cassette or other container into a reception bay of the autoinjector device. The autoinjector drug delivery device can also include a temperature indication capability to provide a visual and/or audio indication to a user when the drug has reached an optimal injection temperature.

The cassette or other container for the drug can include a label, attached or incorporated therein, that is configured to convey the drug's viscosity profile based on temperature to the autoinjector device. The label can be a passive or active device and can provide the viscosity profile directly to the autoinjector device or can provide data allowing the autoinjector device to access or obtain the viscosity profile. The optimum temperature value for a preferred injection viscosity can be room temperature, but alternatively could be a lower temperature, which reduces the wait time to for the drug to reach the injection temperature and further improves the overall patient experience.

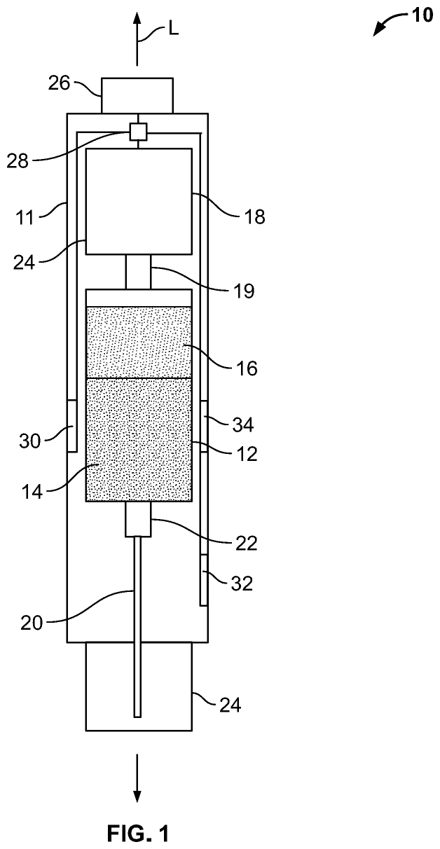

In some versions as illustrated in FIG. 1, drug delivery devices 10, such as autoinjectors, can have a vertically oriented configuration with some or all drug delivery components disposed in stacked relation along a longitudinal axis L within a housing 11 of the devices 10. As a more specific example, the devices 10 can be configured to operate and inject a user with the device 10 oriented generally perpendicular to a skin surface of the user. The drug delivery components can include a reservoir 12 having a drug 14 contained therein, a stopper 16 disposed within the reservoir 12 and sildably movable therein along the longitudinal axis L, a drive mechanism 18 coupled to a plunger 19 to drive the stopper 16 through the reservoir 12, a needle 20 oriented along the longitudinal axis L, a flow path 22 fluidly coupling the reservoir 12 to the needle 20, and a needle insertion mechanism 24 configured to insert the needle 20 to a desired subcutaneous depth within the user. By some approaches, the needle insertion mechanism 24 can be a retractable needle guard to expose the needle 20 or a drive mechanism to longitudinally move the needle a desired distance. For example, the drive mechanism 18 can be configured to drive both movement of the stopper 16 and the needle 20 by moving some or all of the reservoir 12, flow path 22, and needle 20. As commonly configured, one or more of the components of the device 10, such as the drive mechanism 18 and needle insertion mechanism 24, can be operable in response to actuation of a user input device 26 accessible on an exterior of the housing 11. Suitable drive mechanisms include, but are not limited to, springs, gas sources, phase changing materials, motors, or other electromechanical systems. Pursuant to this, the device 10 can include electronic components, such as a controller 28, to control operation of one or more of the drug delivery components. It will be understood that although FIG. 1 shows the components centered along the longitudinal axis L, one or more of the components can be disposed off center from the longitudinal axis L within the housing 11 and still be considered to be in a stacked relation. In one example, an autoinjector drug delivery device having drug delivery components in a stacked relation corresponds to the reservoir 12 co-axially aligned with the needle 20. As described in detail below, the device 10 can include a temperature sensor 30 that is coupled to the controller 28 and is positioned and/or oriented to measure a current temperature of at least one of the reservoir 12, a sidewall of the reservoir 12, the housing 11, or the drug 14 within the reservoir 12. The device 10 can further include an output device 32 that is configured to output data relating to the current temperature. If desired, the device 10 can also include a reader 34 that is configured to read, scan, or otherwise determine injection data from a drug data portion associated with the reservoir 12 to determine drug delivery data for the drug 14. Example autoinjector devices are described in U.S. Ser. No. 62/447,174, filed Jan. 17, 2017, which is hereby incorporated by reference herein.

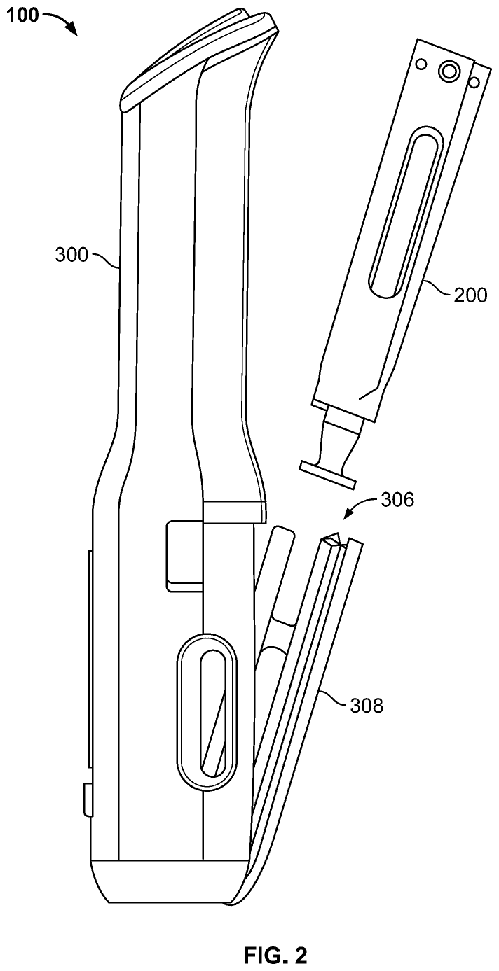

FIG. 2 shows an embodiment of an autoinjector system or apparatus 100 that can be used for injecting a dose of pharmaceutical product (drug) into a patient, the injection often being self-administered by the patient (user). Alternatively, the drug can be administered by a health-care provider. As shown, the autoinjection system or apparatus 100 may comprise a container 201, which in the illustrated form is a removable cassette 200, and one example autoinjector drug delivery device 300. Various embodiments of the cassette 200 may be constructed to contain a drug to be injected into the user by the autoinjector 300. In various other embodiments the cassette 200 may be constructed for use in training the user to operate the autoinjector 300 (a training cassette). The autoinjector 300 may be constructed to deliver an injection automatically upon actuation by the user or some other person. Various embodiments of the autoinjector 300 may have a cassette door 308 that can be constructed to pivot between and an open position and a closed position to allow insertion of the cassette 200 into a bay of a cassette receiving section 306 of the autoinjector 300 and removal of the cassette 200 from the cassette receiving section 306. In some embodiments, the cassette door 308 may include a “cassette” icon (not shown) that indicates the insertion entry point for the cassette 200.

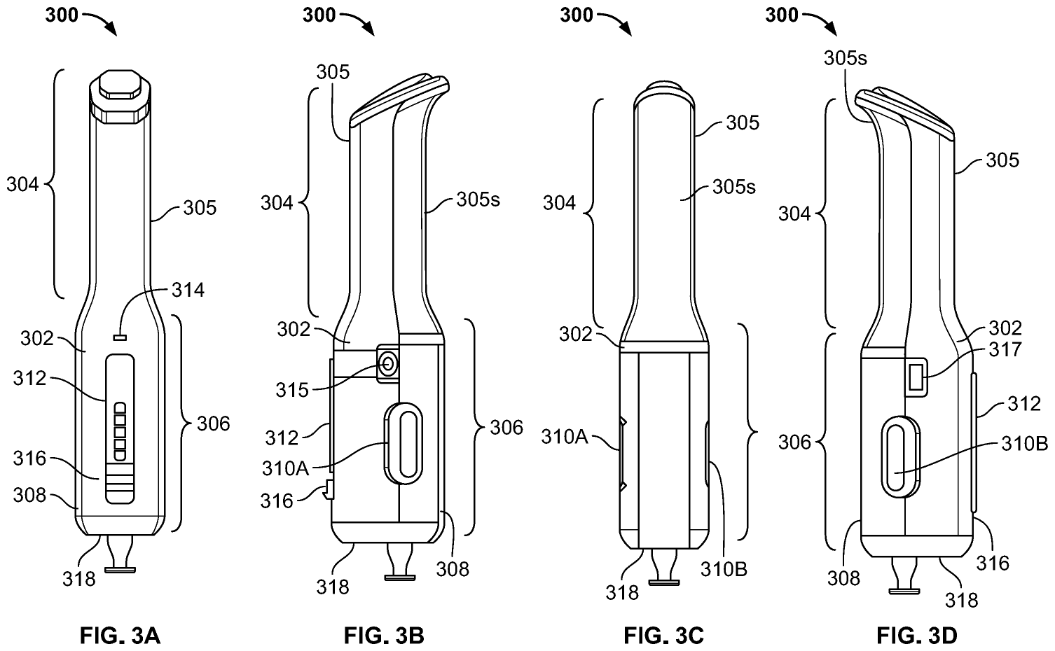

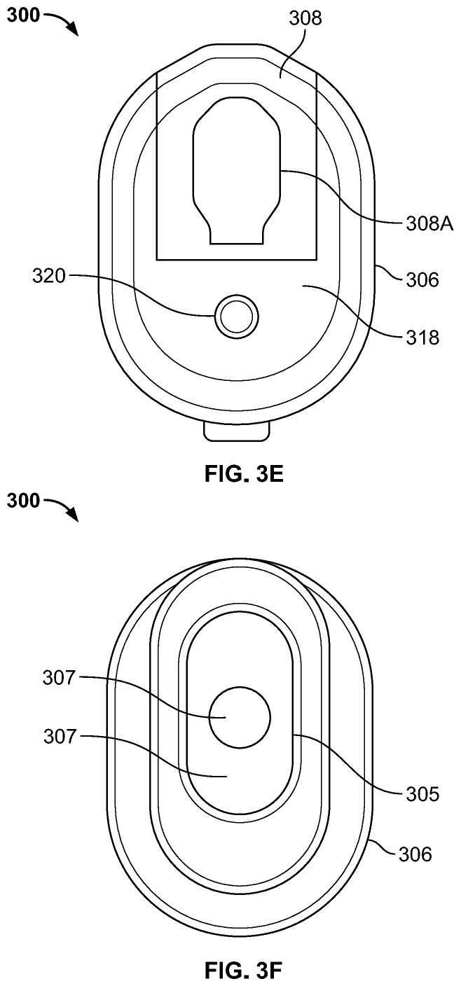

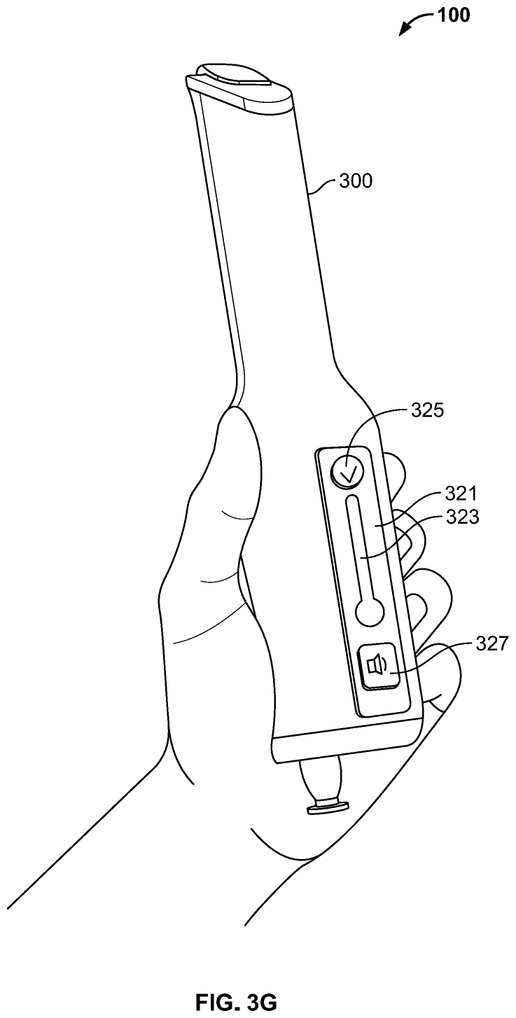

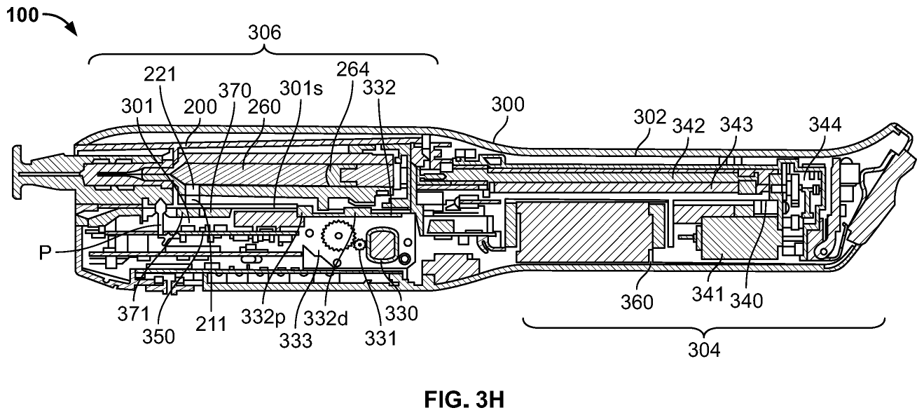

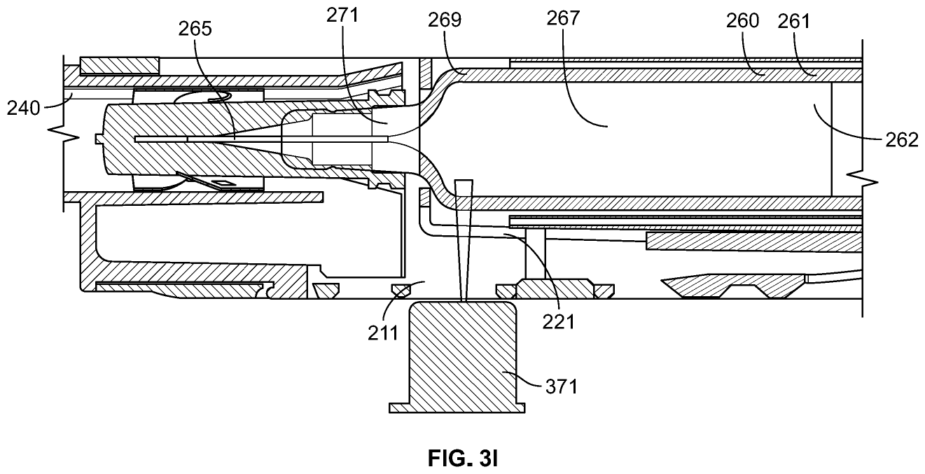

Referring collectively to FIGS. 3A-3I, various embodiments of the autoinjector 300 may comprise a casing 302 having a handle section 304 and a cassette receiving section 306 inline with the handle section 304. To aid patients with manual dexterity issues, the handle section 304 of the autoinjector casing 302 may define an ergonomically shaped handle 305 with a soft grip area 305S. The cassette receiving section 306 comprises the cassette door 308 (FIGS. 3B and 3D) described earlier. The cassette door receives the cassette 200 in an open position (FIG. 2) and aligns the cassette 200 with insertion and extrusion drives, and other structures and components of the autoinjector 300 in a closed position. The cassette door 308 may include a “cassette” icon that indicates the insertion entry point for the cassette 200. The cassette receiving section 306 of the casing 302 may comprise windows 310A, 310B on sides thereof that align with windows of the cassette 200 when the cassette door 308 is closed with the cassette 200 correctly installed therein. In one or more embodiments, the windows 310A, 310B may be double-layered. One or more lights (not shown) may be provided inside the casing 302 to evenly backlight illuminate the cassette windows 212 and the syringe 260 disposed within the inner sleeve 220 of the cassette 200, so that the user can observe the injection cycle through the windows 310A, 310B of the autoinjector 300, i.e., observe the initial and end positions of the plunger-stopper 264 of the syringe 260 during the syringe content (hereinafter “drug”) extrusion process, as well as syringe movements within the cassette 200.

Referring still to FIGS. 3A, 3B, 3D, 3F, and 3G, the autoinjector 300 may further comprise a user interface 312 and an audio speaker (not shown). The user interface 312 (best illustrated in FIG. 3A) may be located in the cassette receiving section 306 of the casing 302, and provides various visual indicators. The audio speaker may be disposed inside the casing 302 and provide various audible indicators. The audio speaker may audibly communicate with the external environment via a speaker aperture 314 formed in the casing 302 in the cassette receiving section 306. The visual and audible indicators generated by the user interface 312 and the audio speaker can tell the user when the autoinjector 300 is ready for use, the progress of the injection process, injection completion, the occurrence of any errors, and other information. The autoinjector 300 may further comprise one or more of a settings/mute switch 315, a speed selector switch 316, a start button 307, and an eject button 317. The settings/mute switch 315 (FIG. 3B) may be located in the cassette receiving section 306 of the casing 302. The mute switch 315 may be constructed allow the user to turn on and off all synthesized sounds, except error sounds, and to respond in real-time so that if the user begins the injection process and changes the mute switch to off, the sounds are immediately muted. The mute switch 315 may also be constructed to slide toward a “mute” icon to mute the audio speaker. A light indicator may be provided to confirm the “mute” state. The speed selector switch 316 (FIGS. 3A and 3B) may be located in the cassette receiving section 306 of the casing 302. The speed selector switch 316 may be constructed to allow the user to select among a plurality of preset drug delivery (extrusion) speeds to accommodate personal patient preference. The speed selector switch 316 may comprise a three switch positions. Other embodiments of the speed selector switch may comprise two switch positions, or 4 or more switch positions. In still other embodiments, the speed selector switch may be of the infinitely variable type. In some embodiments, changing the position of the switch 316 prior to injection changes the speed of drug extrusion during injection while changing the position of the speed selector switch 316 during injection, does not change the speed of the injection in real time. The autoinjector 300 may also be provided with one or more demo cassettes to allow the user to experiment with different speeds of drug delivery. The start button 307 may be disposed at a free end of the handle 305. The button 307 may include an indentation 3071 (FIG. 3F) for optimizing thumb placement on the button 307. The button 307 may be made of a translucent material that allows a lighting effect to illuminate the button as signals. The eject button 317 (FIG. 3D) may be located in the cassette receiving section 306 of the casing 302. The eject button 317 may include an indentation 3171 for optimizing finger placement on the button 317. In some embodiments, the eject button 317 may be controlled by a microprocessor 350 (FIG. 3H) of the autoinjector 300, which may be programmed to eliminate accidental inputs during the injection process.

Referring to FIG. 3E, the cassette receiving section 306 of the casing 302 and the cassette door 308 may form a proximal end wall 318 of the autoinjector 300. The proximal end wall 318 may be configured as a broad, flat and stable base for easily positioning the autoinjector 300 on a support surface, after removal of the shield remover 240 or when the autoinjector 300 does not contain the cassette 240. The portion of the proximal end wall 318 formed by the cassette door 308 may include an aperture 308A that is sized and shaped to allow the shield remover 240 to be removed from the cassette 200 and withdrawn through the aperture 308A, when the cassette 200 is installed in the autoinjector 300. The proximal end wall of the autoinjector 300 may further comprise a target light 320. The target light 320 may be constructed to turn on when the shield remover 240 is removed from the cassette 200 and withdrawn through the aperture 308A, thereby visually indicating that the shield remover 240 has been removed. Once turned on, the target light aids the user in visualizing and selecting an injection site.

As shown in FIG. 3H, various embodiments of the autoinjector 300 may comprise a chassis 301 disposed in the casing 302 for supporting a motorized needle insertion drive 330, a motorized drug extrusion drive 340, a controller 350, a battery 360 for powering the drives 330, 340 and the controller 350, and the skin sensor 380. The term controller refers broadly to any microcontroller, computer, or processor-based device with processor, memory, and programmable input/output peripherals, which is generally designed to govern the operation of other components and devices. It is further understood to include common accompanying accessory devices, including memory, transceivers for communication with other components and devices, etc. These architectural options are well known and understood in the art and require no further description here. The controller 350 may be configured (for example, by using corresponding programming stored in a memory as will be well understood by those skilled in the art) to carry out one or more of the steps, actions, and/or functions described herein.

The casing 302 may define an ergonomically shaped handle section 304 and a cassette receiving section 306. The chassis 301 may include a support surface 301s for supporting one or more cassettes 200 in the autoinjector 300 and aligning the cassette 200 or a selected one of the one or more cassettes 200 with motorized needle insertion and drug extrusion drives 330 and 340, respectively. The insertion drive 330 may include an insertion rack 332, an insertion drive motor 331 and an insertion drive gear train 333 for transmitting rotary motion of the insertion drive motor 331 to drive the rack 332. The insertion rack may include a tab arrangement including, for example, proximal and distal tabs 332p and 332d, respectively, which interface with the cassette 200. The extrusion drive 340 may comprise an extrusion drive motor 341, a plunger rod 342, a lead screw 343, and an extrusion drive gear train 344. The plunger rod 342 is driven by the extrusion drive motor 341 through the lead screw 343 and the extrusion drive gear train 344, and may interface with a plunger 264 of a drug container 260 contained within the cassette 200. The autoinjector 300 can be used for executing multiple injections.

Referring still to FIG. 3H, the controller 350 of the autoinjector 300 may be programmed with instructions that, when executed by the controller 350, enable it to control and monitor the various operations and functions of the autoinjector 300. For example, but not limitation, the controller 350 may be programmed with instructions for controlling the motorized insertion and extrusion drives 330, 340. Such instructions may control and monitor each step of the injection cycle and process flow, thereby automating needle insertion, drug extrusion, and needle retraction, and controlling the sequence of actions performed by the user so that the injection process and drug administration can be made more reliable, accurate, and consistent. The controller 350 may also be programmed with instructions for controlling the audible and visual feedbacks to the user. An automated power-on self-test checks the operation of the autoinjector 300 and remaining battery charge.

In various other embodiments, the autoinjector 300 may include other types of needle insertion drives, drug extrusion drives, and means for activating and sequencing the drives. The insertion and extrusion drives, in such embodiments may be implemented as separate and distinct mechanisms or combined into a single mechanism. The insertion and extrusion drives of such embodiments may be powered, without limitation, by motors, mechanical mechanisms (e.g., elastic members such as springs), gas pressure mechanisms, gas releasing mechanism, or any combination thereof. Various transmission mechanisms may be used for transmitting the power to the cassette, to cause injection of the drug. In addition, the activating and sequencing means may comprise various mechanical and electromechanical arrangements, which may be combined with the controller described earlier or used alone. The autoinjector in such embodiments may be constructed to be reusable for executing multiple injections or be designed for a single, disposable use.

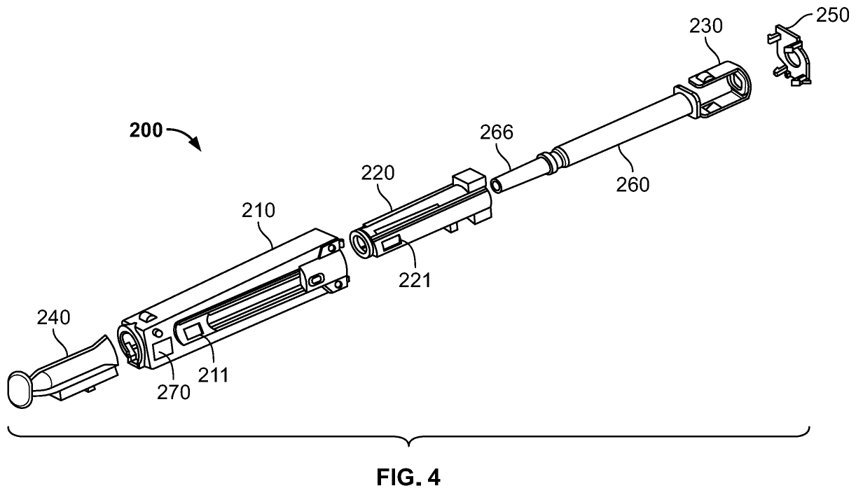

Referring now to FIG. 4, various embodiments of the cassette 200 may comprise an outer housing 210, an inner sleeve 220, a drug container 260 for containing a drug, a cassette cap 240, a lock cap 230, and a cover 250. Such embodiments of the cassette 200 facilitate and enable easy injection of the drug with the autoinjector and can be constructed for a single, disposable use. As shown, the outer housing 210 and inner sleeve 220 can define windows 211, 221, respectively, providing visual access to the drug container 260 to thereby determine a temperature of a drug contained within the container 260, as described in more detail below. The windows 211, 221 can be openings extending through the outer housing 210 and inner 220, for example. In various embodiments, the lock cap 230 and cover 250 of the cassette 200 may be constructed to resist removal of the drug container 260 from the cassette 200, thereby preventing needle sticks before and after use of the cassette 200 and also preventing the drug container 260 from being taken out of the cassette 200 or replaced. In addition, the lock cap 230 and cover 250 protect the drug container 260 during shipment and transportation. The cassette cap 240, in various embodiments, may be constructed to remove a needle shield 266 covering an injection needle associated with the drug container 260. In various other embodiments, the cassette cap 240 may also be constructed to engage the outer housing 210 of the cassette 200, such that the cassette cap 240 cannot be rotated or twisted, thereby preventing the needle shield 266 from damaging the injection needle. Various embodiments of the inner sleeve 220 may be constructed to position the drug container 260 within the cassette housing 210 in either a needle-concealed position or a needle injection position during an injection cycle of the autoinjector. In various other embodiments, the outer housing 210 and the inner sleeve 220 of the cassette 200 may include one or more locking arrangements that protect the drug container 260 and prevent unintended needle exposure or damage.

The container 201 may include an identification arrangement that interfaces with the autoinjector 300 to communicate the installation of the container 201 within the autoinjector 300, information about the container 201, and/or data about contents of the container 201. In one version, the container 200 further includes a drug data portion 270 that is configured to provide drug delivery data to the autoinjector 300 associated with a drug 267 within the container 200. In some examples, the drug delivery data can include an optimum drug delivery temperature, which can be a particular temperature, a range of temperatures, or a threshold temperature, corresponding to the drug 267 having a viscosity suitable for injection, an identification of the drug 267, an age of the drug 267, and so forth.

The drug data portion 270 can take any suitable form capable of providing the delivery data to the autoinjector 300. In a first form, the drug data portion 270 can be a machine readable code, such as a QR code, UPC code, etc., capable of being scanned and read by the autoinjector 300. In other versions, the drug data portion 270 can be a radio frequency identification (RFID) tag capable of being read by the autoinjector 300 or sending the drug delivery data to the autoinjector 300 or a tactile or visual code capable of being deciphered by the autoinjector 300.

As shown in FIG. 3H, the autoinjector 300 can include a reader 370 configured to read, scan, or otherwise interact with the drug data portion 270 to obtain the delivery data. The reader 370 can be mounted to the casing 302, provided on or in the cassette support surface 301s, or in other suitable locations on or adjacent to the cassette receiving section 306. The reader 370 may be coupled with the controller 350 in a manner that allows signals or data to be communicated to the controller 350. In some versions, the reader 370 can be a scanner configured to reader a machine readable code or RFID tag.

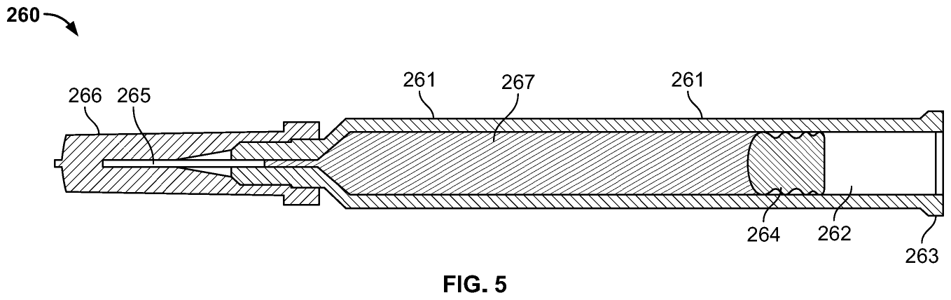

As shown in FIG. 5, the drug container 260 may comprise a conventional glass or plastic syringe comprising a barrel 261 that defines a fluid chamber or reservoir 262. The fluid chamber 262 may be filled for treatment or be prefilled with a predetermined dose of a drug 267. The drug 267 may have a viscosity that depends on the temperature of the product. The syringe 260 may further comprise an injection needle 265 removably or fixedly disposed at a proximal end of the barrel 261, and an outwardly extending flange 263 disposed at a distal end of the barrel 261. The injection needle 265 may communicate with the fluid chamber 262 to allow dispensing of the predetermined dose of the drug 267 expelled from the fluid chamber 262 of the syringe barrel 261. The syringe 260 may further comprise a moveable plunger-stopper 264, disposed within the fluid chamber 262 of the barrel 260, for expelling the predetermined dose of the drug 267 from the chamber 261 so that it may be dispensed through the injection needle 265. A protective needle shield 266 made, for example, of a non-rigid material, may be provided for covering the injection needle 265.

In some embodiments, the drug contained in the drug container 260 may have a viscosity of about 19 centipoise, at room temperature (20 to 25° C. [68-77° F.]). In some embodiments, the drug contained in the drug container 260 may have a viscosity ranging between about 1 centipoise and about 320 centipoise, at room temperature. In some embodiments, the drug contained in the drug container 260 may have a viscosity ranging between about 5 centipoise and about 40 centipoise, at room temperature. In some embodiments, the drug contained in the drug container 260 may have a viscosity ranging between about 10 centipoise and about 35 centipoise, at room temperature. In some embodiments, the drug contained in the drug container 260 may have a viscosity ranging between about 15 centipoise and about 30 centipoise, at room temperature. In some embodiments, the drug contained in the drug container 260 may have a viscosity ranging between about 20 centipoise and about 25 centipoise, at room temperature. In some embodiments, the drug contained in the drug container 260 may have a viscosity ranging between about 16 centipoise and about 42 centipoise, at room temperature. In some embodiments, the drug contained in the drug container 260 may have a viscosity ranging between about 1 centipoise and about 29 centipoise, at room temperature.

As shown in FIGS. 3H and 3I, the autoinjector 300 can further include a temperature sensor 371 that is configured to determine a current temperature of at least one of the reservoir 262, a sidewall of the reservoir 262, the housing 302 of the device 300, the drug 267 within the reservoir 262, or other structure or surfaces of the container 260. In one example, the temperature sensor 371 can be disposed in or adjacent to the bay of the cassette receiving section 306 so that the container 201 is aligned with the temperature sensor 371 when the container 201 is inserted into the autoinjector 300. For example, the temperature sensor 371 can be mounted to the casing 302, provided on or in the cassette support surface 301s, or in other suitable locations. In the illustrated form, the temperature sensor 371 is disposed adjacent to the end wall 318 of the autoinjector 300 so as to align with a distal end 269 of the drug container 260 proximate to a needle hub 271 thereof. This positioning allows the temperature sensor 371 to align with and measure the temperature of the drug container 260 and the drug 267 received therein regardless of a particular shape of the container or a fill volume of the drug container 260.

The temperature sensor 371 may be coupled with the controller 350 in a manner that allows signals or data to be communicated to the controller 350. The temperature sensor 371 can take any suitable form. In some versions, the temperature sensor 371 can be contactless and capable of measuring the temperature of the drug 267 in a position spaced from the container 201. For example, the temperature sensor 371 can be an infrared temperature sensor, such as an infrared thermopile. Other versions can include a resistance temperature detector, a thermocouple, or an assembly comprising a thermally-sensitive label and an optical detector.

In versions where the temperature sensor 371 is an infrared thermopile chip, the sensor measures the infrared signature of a thermal source without direct contact with the source. These infrared thermopile chips may operate in a wavelength range of 0.7 μm to 1000 μm and may have a footprint of less than 2 mm by 2 mm. Such a sensor may provide a determination of the ambient temperature and, as such, the ambient temperature may be used to predict how long the drug 267 will take to reach the injection temperature. This prediction may rely on the ambient temperature, as well as the thermal mass and thermal transfer properties of the drug 267, which can be included in the delivery data or accessed or obtained in response to reception of the delivery data, and the controller 350 may be programmed to perform the calculation or a reference table may be stored in memory for the processor to access once the ambient temperature is determined. In other versions, the temperature sensor 371 may be a thermally-sensitive label used in conjunction with (coupled to) an optical pickup or sensor. The thermally-sensitive label will change its appearance when a threshold temperature is reached. The label may take the form of a physical label, a wax, a lacquer-like paint, or a liquid crystal polymer film, for example. The optical pickup can be used to determine this change in appearance, which can then be correlated with the threshold temperature to make a temperature determination. The optical pickup may have a footprint of less than 2.5 mm by 2.5 mm, and may draw less than 20 μA when in active mode, 0.5 μA when in low-power non-active mode. According to such an embodiment, the coupling between the label and the pickup is non-contact, thus preventing direct physical interaction between the label and the pickup.

As described above with respect to FIG. 4, the outer housing 210 and inner sleeve 220 define windows 211, 221, respectively, which provide visual access to the drug container 260. As shown in FIG. 3I, when the cassette 200 is inserted into the bay of the cassette receiving section 306 and the door 308 is closed, the windows 211, 221 are aligned within the temperature sensor 371.

Further, as shown in FIG. 3G, the user interface 312 described above can include an output device to provide various visual indicators and/or audible indicators. For example, the user interface 312 can include a display and/or an audio speaker. The visual and audible indicators generated by the user interface 312 can tell the user when the autoinjector 300 is ready for use, the progress of the injection process, injection completion, the occurrence of any errors, and other information. In one example, the output device can be configured to output data relating to the current temperature of the at least one of the reservoir 262, a sidewall of the reservoir 262, the housing 302 of the device 300, the drug 267 within the reservoir 262, or other structure or surfaces of the container 260. The data can include one or more of the following: the temperature itself, a binary signal relating to the temperature (such as selectively illuminating an indicator light or differently colored lights, e.g. green and red, to signal that the current temperature is a desired temperature or within a desired range or that the current temperature is not at the desired temperature or outside of the desired range), an audible signal in response to the current temperature reaching a predetermined threshold, a comparison between the current temperature and a target temperature, which can be a range of temperatures (such as within 2 or 4 degrees of a target temperature), an estimated time until the current temperature is expected to reach a threshold temperature, etc.

In a further version shown in FIG. 3G, the user interface 312 can include a drug temperature display 321 providing a visual indication of a current temperature of the drug 267 to a user. In the illustrated form, the drug temperature display 321 includes a scale 323, in the form of a thermometer, configured to visually display the temperature of the drug 267 in relation to a desired injection temperature, which can be a threshold temperature, a range of temperatures, or a specific temperature. Pursuant to this, the controller 350 can access the delivery data obtained by the reader 370 to determine the injection temperature. The controller 350 can then sequentially increase an amount of illumination or display filling up the scale 323 as the drug 267 heats up until the scale 323 is fully illuminated when the temperature of the drug 267 corresponds to the injection temperature. A fully illuminated scale 323 can provide a visual indication to a user that the drug 267 is in a proper state for an injection operation. If desired, the scale 323 can utilize a color gradient to provide additional feedback to a user. For example, the scale 323 can have cooler colors, e.g., starting with blue, at the low end thereof and sequentially transition to warm colors, e.g., finishing with orange or red. Further, the user interface 312 can include a ready state indicator 325, such as a check mark as shown, that can be illuminated or otherwise displayed by the controller 350 when the temperature of the drug 267 corresponds to the injection temperature. Finally, as discussed above, the audio speaker may audibly communicate with the external environment via the speaker aperture 314 formed in the casing 302 in the cassette receiving section 306 to provide audible indicators generated by the user interface 312 to tell the user when the autoinjector 300 is ready for use when the temperature of the drug 267 corresponds to the injection temperature. Pursuant to this, the user interface 312 can include a volume setting switch 327, which can increase, decrease, and/or mute the audible communications.

So configured, the controller 350 of the autoinjector 300 is coupled to the temperature sensor 371, the reader 370, and the output devices of the user interface 312. The controller 350 is programmed to receive a signal including the delivery data from the reader 370 and determine an injection temperature for the drug 267 within the container 201, such as by receiving the injection temperature or by reference to a stored or remote table. The controller 350 is also programmed to receive signal indicating a current temperature measurement of the drug 267 from the temperature sensor 371 and compare the current temperature of the drug 267 with the injection temperature. Thereafter, the controller 350 is programmed to control operation of the output devices of the user interface 312 to provide a visual or audio indication to a user of the autoinjector 300 of the result of the comparison as described above indicating whether the temperature of the drug 267 is lower than the injection temperature or corresponds to the injection temperature. The controller 350 can also be programmed to lock operation of the autoinjector 300 in response to determining that the temperature of the drug 267 is below (or above) the injection temperature.

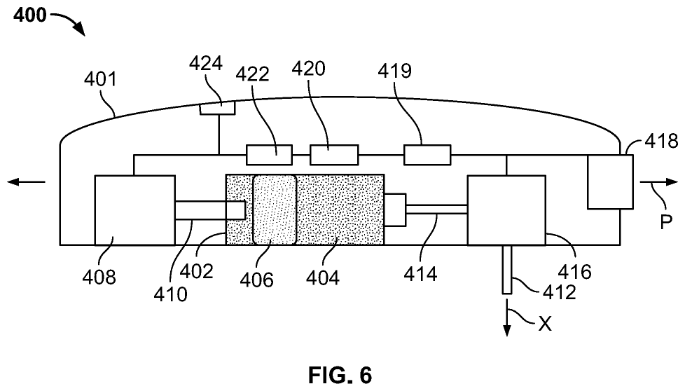

Although the above disclosure has been described with reference to the structure and operation of autoinjector drug delivery devices, the disclosure is also suitable for and can be incorporated within on body drug delivery devices. As illustrated in FIG. 6, on body injectors 400 can have a horizontally oriented configuration with drug delivery components disposed generally along a horizontal plane P within a housing 401 of the devices 400. With these devices 400, the housing 401 has a low profile with a larger width than height so that when a user positions the housing 401 on the skin, the components are spread out over an area of the skin rather than stacked as with the above embodiments. The drug delivery components can include a reservoir 402 having a drug 404 contained therein, which can be removably disposed within the housing 401, a stopper 406 disposed within the reservoir 402 and sildably movable therein along the horizontal plane P, a drive mechanism 408 coupled to a plunger 410 to drive the stopper 406 through the reservoir 402, a needle 412 oriented along an axis X that extends generally perpendicular to the horizontal plane P, a flow path 414 fluidly coupling the reservoir 402 to the needle 412, and a needle insertion mechanism 416 configured to insert the needle 412 to a desired subcutaneous depth within the user. As commonly configured, one or more of the components of the device 400, such as the drive mechanism 408 and needle insertion mechanism 416, can be operable in response to actuation of a user input device 418 accessible on an exterior of the housing 401. Pursuant to this, the device 400 can include electronic components, such as a controller 419, to control operation of one or more of the drug delivery components. Of course, it will be understood that some components can be disposed partially or entirely above or below the horizontal plane P extending generally centrally through the housing 401 and still be considered to have a horizontally oriented configuration. Suitable drive mechanisms include, but are not limited to, springs, gas sources, phase changing materials, motors, or other electromechanical systems. In these versions, the device 400 can include a temperature sensor 420 and reader 422 having an operation similar to that described above with respect to the autoinjector embodiments. The temperature sensor 420 is coupled to the controller 419 and is positioned and oriented to measure a current temperature of at least one of the reservoir 402, a sidewall of the reservoir 402, the housing 401, or the drug 404 within the reservoir 402. The reader 422 can be configured to read, scan, or otherwise determine injection data from a drug data portion associated with the reservoir 402 to determine drug delivery data for the drug 404. The device 400 can further include an output device 424 that is configured to output data relating to the current temperature. Example on body injector devices are described in U.S. Ser. No. 62/536,911, filed Jul. 25, 2017, which is hereby incorporated by reference herein.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIIa receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

It will be appreciated that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. The same reference numbers may be used to describe like or similar parts. Further, while several examples have been disclosed herein, any features from any examples may be combined with or replaced by other features from other examples. Moreover, while several examples have been disclosed herein, changes may be made to the disclosed examples within departing from the scope of the claims.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing;
a reservoir removably disposed within the housing and having a sidewall defining an interior for containing a drug;
a temperature sensor configured to measure a current temperature relating to at least one of the reservoir, the sidewall of the reservoir, the housing, or the drug; and
an output device configured to output data relating to the current temperature.

2. The drug delivery device of claim 1, further comprising a controller coupled to the temperature sensor and the output device, the controller being programmed to compare the current temperature to a predetermined target temperature.

3. The drug delivery device of claim 2, wherein the controller is further programmed to receive and/or output data relating to the current temperature and the predetermined target temperature.

4. The drug delivery device of claim 2, wherein the controller is further programmed to provide a signal to a user in response to determining that the current temperature corresponds to the predetermined target temperature.

5. The drug delivery device of claim 1, further comprising a drug data portion associated with the reservoir.

6. The drug delivery device of claim 5, further comprising a reader configured to read the drug data portion to determine injection data associated with the drug.

7. The drug delivery device of claim 6, further comprising a controller coupled to the reader and the temperature sensor, the controller being programmed to receive input from the reader and to compare the current temperature to a predetermined target temperature from the reader.

8. The drug delivery device of claim 6, wherein the drug data portion comprises a machine readable code; and the reader comprises a scanner configured to read the machine readable code.

9. The drug delivery device of claim 1, wherein the housing further comprises a bay configured to removably receive the reservoir therein.

10. The drug delivery device of claim 9, wherein the temperature sensor is disposed adjacent to the bay.

11. The drug delivery device of claim 9, wherein the housing comprises a door movable between an open position and a closed position, the door configured to receive the reservoir in the open position and align the reservoir with the temperature sensor within the bay in the closed position.

12. The drug delivery device of claim 1, further comprising a cassette configured to receive the reservoir therein, the cassette defining a window providing access for the temperature sensor to measure the current temperature.

13. The drug delivery device of claim 1, wherein the output device comprises a display including a scale portion providing a visual indication of the current temperature.

14. The drug delivery device of claim 1, further comprising the drug disposed in the reservoir.

15. A method of delivering a drug product, the method comprising:
receiving a reservoir adapted to contain a drug within a drug delivery device;
measuring a current temperature relating to at least one of the reservoir, a sidewall of the reservoir, a housing of the drug delivery device, or a drug contained within the reservoir with a temperature sensor; and
outputting data with an output device of the drug delivery device relating to the current temperature.

16. The method of claim 15, further comprising reading a drug data portion associated with the reservoir with a reader of the drug delivery device to determine injection data associated with the drug contained within the reservoir.

17. The method of claim 16, further comprising:
determining an injection temperature for the drug associated with the injection data with a controller of the drug delivery device; and
determining whether the current temperature corresponds to the injection temperature with the controller.

18. The method of claim 17, wherein the output device comprises a display; and outputting data comprises displaying a visual indication of the current temperature in relation to the injection temperature on a scale portion of the display.

19. The method of claim 16, wherein reading the drug data portion with the reader of the drug delivery device comprises reading a machine readable code with a scanner.

20. The method of claim 15, wherein receiving the reservoir within the drug delivery device comprises receiving a cassette including the reservoir in a bay of an autoinjector device.
